(19) United States
(12) Patent Application Publication
(10) Pub. No.: US 2026/0284361 A1
(43) Pub. Date: Sep. 24, 2026

(54) INTRAVASCULAR DEVICE FOR INTRODUCING MEDICAL DEVICES INTO VASCULAR SYSTEMS

(71) Applicant: Heraeus Medical Components LLC, Fridley, MN (US)

(72) Inventors: Jack DANT, Fridley, MN (US); Drew SCHILTZ, Fridley, MN (US); Patrick OLSON, Fridley, MN (US)

(73) Assignee: Heraeus Medical Components LLC, Fridley, MN (US)

(21) Appl. No.: 19/082,996

(22) Filed: Mar. 18, 2025

(51) Int. Cl. A61M 25/06

(52) U.S. Cl. CPC A61M 25/0662; A61M 2025/0681; A61M 2207/00

(57) ABSTRACT

One aspect relates to an intravascular device for introducing medical devices into vascular systems, including a hollow cylindrical inner liner with a proximal end, a distal end, and an inner lumen extending axially through the inner liner between the proximal end and the distal end. The inner lumen is surrounded by an inner liner inner surface of the inner liner. An outer jacket is arranged coaxially around the inner liner. The inner liner inner surface has a plurality of ribs extending axially from the proximal end to the distal end of the inner liner. One aspect also relates to a method for manufacturing such an intravascular device.

### DESCRIPTION

One aspect relates to an intravascular device for introducing medical devices into vascular systems, comprising:
a hollow cylindrical inner liner comprising a proximal end, a distal end, and an inner lumen extending axially through the inner liner between the proximal end and the distal end, wherein the inner lumen is surrounded by an inner liner inner surface of the inner liner, an outer jacket arranged coaxially around the inner liner, and
wherein the inner liner inner surface comprising a plurality of ribs extending axially from the proximal end to the distal end of the inner liner.

Aspects also relates to a method for manufacturing such an intravascular device.

### BACKGROUND OF THE INVENTION

Among the most significant medical and surgical advances in recent decades has been the introduction and now routine implementation of a large number of minimally invasive procedures. These procedures include angioplasty, endoscopy, laparoscopy, and arthroscopy along with numerous other diagnostic and therapeutic interventions. Such minimally invasive procedures differ from conventional open surgical procedures in that access to a specific site in the patient is gained via a small incision into which a tubular device, or at least part of it, is inserted or introduced. The tubular device, or the tubular part of the device, keeps the incision open and at the same time enables access to the vascular system of the patient via a cavity inside the device. A particularly significant example of a minimally invasive technique is the temporary or permanent implantation of a medical intervention device, such as a stent, into a vessel of a patient.

Other examples relate to the transfer of a liquid medication to a target area and/or the removal of body fluid from the vascular system.

Intravascular devices such as catheters and introducer sheaths are typically used when performing these and other desired techniques-particularly when introducing medical devices.

However, these intravascular devices tend to kink-in particular, when introduced into angled parts of the vascular system of the patient. Kinking reduces the effective inner diameter of the intravascular devices, which often makes it unusable. At the same time, the intravascular devices must be highly flexible-in particular, to be able to be introduced into the narrower vessels of the patient.

In order to be able to be introduced into narrower vessels and at the same time ensure the largest possible inner lumen for the feeding through of intervention devices, intravascular devices often have thin walls, wherein the wall is equipped with a reinforcement structure, like a braid or coil, for structural stabilization in order to achieve high resistance to kinking at the same time. Such an intravascular devices is described, for example, in U.S. Pat. No. 5,700,253 A.

However, such reinforcement structures should not be exposed to the inner lumen of the intravascular device, since this would make it more difficult for medical devices to feed through the inner lumen of the intravascular devices-for example, due to increasing friction or getting stuck. Therefore, the inside of the intravascular devices usually has a inner liner made of a polymer, which is intended to enable medical devices to feed through the intravascular device as easily and smoothly as possible. The inner liner should have the lowest possible coefficient of friction in order to enable such devices to be introduced smoothly through the inner liner of the intravascular device. Polytetrafluoroethylene-based (PTFE) coatings predominate on the market.

However, due to the potential environmental impact of PTFE materials, there is a desire in the market for a suitable replacement for PTFE-based inner liner.

### OBJECTS

One object of the present embodiments is to overcome, at least in part, one or more of the disadvantages resulting from the prior art.

One aspect is based upon the objective of providing an intravascular device, especially in the form of a catheter or an introducer sheath—in particular, an inner liner of an intravascular device—which enables medical devices to feed through the inner lumen of the intravascular device as smoothly, easily, and in as a trouble-free manner as possible. Furthermore, the intravascular device should be designed to be as flexible as possible, such that introduction is enabled even in narrower and more angled vascular systems of a patient. The intravascular devices should comprise materials that are as environmentally compatible as possible; in particular, the introducer sheath should be constructed free of materials containing polytetrafluoroethylene. The material of the inner liner of the intravascular devices should be compatible with the biocompatible materials commonly used on the market for conventional intravascular devices, in order to be able to use standard materials and processes. It should be possible to provide the inner liner with the simplest possible processes. The intravascular devices should be mechanically robust.

It is a further object of one aspect to provide a method for manufacturing such an intravascular device, by means of which at least some of the objects already described are at least partially achieved.

### PREFERRED EMBODIMENTS OF THE INVENTION

A contribution to the at least partial fulfillment of at least one of the aforementioned objects is made by the features of the independent claims. The dependent claims provide preferred embodiments that contribute to at least partial fulfillment of at least one of the objects.

A first embodiment is an intravascular device for introducing medical devices into vascular systems, comprising:
a hollow cylindrical inner liner comprising a proximal end, a distal end, and an inner lumen extending axially through the inner liner between the proximal end and the distal end, wherein the inner lumen is surrounded by an inner liner inner surface of the inner liner, an outer jacket arranged coaxially around the inner liner, and
wherein the inner liner inner surface comprising a plurality of ribs extending axially from the proximal end to the distal end of the inner liner.

In one embodiment of the intravascular device, the inner liner comprises a non-fluorinated thermoplastic polymer. This embodiment is a second embodiment of the invention, which preferably depends upon the first embodiment of the invention.

In one embodiment of the intravascular device, the inner liner inner surface comprises 6 to 30 ribs extending axially from the proximal end to the distal end of the inner liner. This embodiment is a third embodiment of the invention, which preferably depends upon the first or second embodiment of the invention.

In one embodiment of the intravascular device, the ribs have a height that is between 0.012 mm and 0.08 mm. This embodiment is a fourth embodiment of the invention, which preferably depends upon one of the forementioned embodiments of the invention.

In one embodiment of the intravascular device, the ribs are evenly spaced around the circumference of the inner liner inner surface. This embodiment is a fifth embodiment of the invention, which preferably depends upon one of the forementioned embodiments of the invention.

In one embodiment of the intravascular device, a maximum distance between two adjacent ribs is 0.5 mm. This embodiment is a sixth embodiment of the invention, which preferably depends upon one of the forementioned embodiments of the invention.

In one embodiment of the intravascular device, the ribs are integrally formed with the inner liner. This embodiment is a seventh embodiment of the invention, which preferably depends upon one of the forementioned embodiments of the invention.

In one embodiment of the intravascular device, the ribs are parallel to each other. This embodiment is a eighths embodiment of the invention, which preferably depends upon one of the forementioned embodiments of the invention.

In one embodiment of the intravascular device, the ribs extend in a straight or spiral pattern along the inner liner inner surface. This embodiment is a ninth embodiment of the invention, which preferably depends upon one of the forementioned embodiments of the invention.

In one embodiment of the intravascular device, the intravascular device further comprising a reinforcement structure, for example a braid or a coil, positioned between the inner liner and the outer jacket. This embodiment is a tenth embodiment of the invention, which preferably depends upon one of the forementioned embodiments of the invention.

In one embodiment of the intravascular device, the inner liner is made of a polymer selected from the group consisting of polyether block amide copolymers, polyamides, polyethylenes and polyimides. This embodiment is an eleventh embodiment of the invention, which preferably depends upon one of the forementioned embodiments of the invention.

A twelfth embodiment of the invention is a method of manufacturing an intravascular device with ribs, preferably an intravascular device according to one of the first to twelfth embodiment of the invention, comprising the method steps:
(a) extruding a hollow cylindrical inner liner having a proximal end, a distal end, and an inner lumen extending axially through the inner liner between the proximal end and the distal end, wherein the inner lumen is surrounded by an inner liner inner surface;
(b) simultaneously forming a plurality of ribs on the inner liner inner surface during the extrusion process, the ribs extending axially from the proximal end to the distal end of the inner liner; and
(c) arranging an outer jacket coaxially around the inner liner.

In one embodiment of the method, arranging the outer jacket comprises a reflow process using a structured mandrel placed in the inner lumen, the structured mandrel having a pattern corresponding to the ribs on the inner liner, thereby preserving the ribs on the inner liner inner surface during the reflow process. This embodiment is a thirteenth embodiment of the invention, which preferably depends upon the twelfth embodiment of the invention.

A fourteenth embodiment of the invention is a method of manufacturing an intravascular device with ribs, preferably an intravascular device according to one of the first to twelfth embodiment of the invention, comprising the method steps:
(a) providing a hollow cylindrical inner liner having a proximal end, a distal end, and an inner lumen extending axially through the inner liner between the proximal end and the distal end, wherein the inner lumen is surrounded by an inner liner inner surface;
(b) placing a structured mandrel in the inner lumen, the structured mandrel having a pattern corresponding to the desired ribs;
(c) heating the inner liner to a temperature sufficient to cause the inner surface to melt and conform the pattern of the structured mandrel, thereby forming a plurality of ribs on the inner liner inner surface, the ribs extending axially form the proximal end to the distal end of the inner liner; and
(d) arranging an outer jacket coaxially around the inner liner.

In one embodiment of the method, arranging the outer jacket comprises a reflow process using the structured mandrel placed in the inner lumen, thereby preserving the ribs on the inner liner inner surface during the reflow process. This embodiment is a fifteenths embodiment of the invention, which preferably depends upon the fourteenth embodiments of the invention.

### GENERAL

In addition to the embodiments described herein, the elements of which “contain” or “comprise” a particular feature (e.g. a material), a further embodiment is always contemplated in which the element in question consists solely of the feature, i.e., does not comprise any other components. The word “comprise” or “comprising” is herein used synonymously with the word “contain” or “containing”.

When in an embodiment an element is referred to in the singular, an embodiment is also contemplated in which a plurality of these elements are present. The use of a term for an element in the plural generally also includes an embodiment in which only a single corresponding element is included.

Unless otherwise stated or clearly excluded from the context, it is fundamentally possible and is hereby clearly considered that features of different embodiments can also be provided in the other embodiments described herein. It is also generally contemplated that all features described herein in conjunction with a method are also applicable for the products and devices described herein, and vice versa.

Merely for the sake of conciseness, these considered combinations are not all explicitly listed in all cases. Technical solutions that are known to be equivalent to the features described herein should be included in principle by the scope of the invention.

In the present description, specifications of ranges also contain the values specified as limits. A specification of the type “in the range from X to Y” with respect to a quantity A consequently means that A can take the values X, Y and values between X and Y. One-sidedly limited ranges of the type “up to Y” for a size A accordingly mean as a value Y and less than Y.

Some of the features described are associated with the term “substantially.” The term “substantially” is to be understood in such a way that, under real conditions and manufacturing techniques, a mathematically exact interpretation of terms such as “superimposition,” “perpendicular,” “diameter” or “parallelism” can never be given exactly, but only within certain manufacturing error tolerances. For example, “substantially perpendicular axes” enclose an angle of 85 degrees to 95 degrees relative to one another, and “substantially equal volumes” comprise a variation of up to 5% by volume. For example, a “device consisting substantially of plastic” comprises a plastic content of ≥95 to ≤100% by weight. For example, a “substantially complete filling of a volume B” comprises a filling of ≥95 to ≤100% by volume of the total volume of B.

Terms such as “proximal” and “distal” merely serve to designate the spatially opposite ends of the device or other structural units of the device, and do not allow any conclusions to be drawn about the orientation in relation to a human body-for example, a user of the device. “Distal to . . . ” and “proximal to . . . ” or similar formulations merely express the spatial arrangement of two structural units of the device in relation to one another.

### DETAILED DESCRIPTION

A first subject matter of one embodiment relates to an intravascular device for introducing medical devices into vascular systems, comprising:
a hollow cylindrical inner liner comprising a proximal end, a distal end, and an inner lumen extending axially through the inner liner between the proximal end and the distal end, wherein the inner lumen is surrounded by an inner liner inner surface of the inner liner, an outer jacket arranged coaxially around the inner liner, and
wherein the inner liner inner surface comprising a plurality of ribs extending axially from the proximal end to the distal end of the inner liner.

The intravascular device is designed to introduce medical devices, such as stents, implants, guidewires or other intravascular devices, into a patient's blood vessels. Preferably, the intravascular device is a catheter, an introducer sheath or a guide sheath for this purpose, the intravascular device has a tube-like body that has at least two coaxial layers, an inner liner and an outer jacket that coaxially surrounds the inner liner. This tube-like body of the intravascular device is introduced at least partially into the blood vessels so that the medical device can be introduced into the patient with the aid of the intravascular device through an inner lumen of the intravascular device that extends axially through the tube-like body.

The inner liner faces the tube-like inner lumen of the intravascular device. Thus, the inner lumen is formed by an inner liner inner surface, facing the longitudinal axis of the intravascular device, in particular the longitudinal axis of the tube-like body of the intravascular device.

Thus, the inner liner comes into direct contact with intervention devices, such as catheters or stents, which are introduced into the vascular system of a patient through the inner lumen.

The material properties of the inner liner, in particular its friction properties, therefore have a direct influence on the insert ability of medical devices through the intravascular device. Therefore, polymers with a particularly favorable friction coefficient, such as fluorinated polymers like polytetrafluoroethylene (PTFE), are usually used for the inner liner.

In order to reduce the friction between a medical device and the inner liner, the inner liner, in particular the inner liner inner surface, according to the invention has a plurality of ribs which extend axially from the proximal end to the distal end of the inner liner. The ribs reduce the contact surface between the medical device and the inner liner inner surface, so that the friction is effectively reduced. The ribs might thus at least partially compensate for unfavorable friction properties of alternative materials to PTFE, even in direct comparison to PTFE. Preferably, the ribs extend over the entire length of the inner liner.

The ribs extend axially and are thus not strictly perpendicular to the longitudinal axis of the inner liner, which reduces the risk of at least partial removal, for example by abrasion, of the ribs by a medical device that is passed through the intravascular device. “Axial” in this context does not mean that the ribs are strictly parallel to the longitudinal axis of the inner liner. For example, the ribs may be at an angle to the longitudinal axis of the inner liner in a range between 0° and 30°, preferably between 3° and 25°, to the longitudinal axis of the inner liner.

The ribs form protrusions of the inner liner inner surface, which protrude into the inner lumen. The ribs may have a polygonal cross-section, for example a triangular or rectangular cross-section. Preferably, the ribs have a rounded cross-section, for example a semicircular cross-section. A rounded cross-section allows for the smallest possible contact area between the inner liner and the medical device and minimizes the risk of the medical device passing through the intravascular device getting stuck between the ribs, which could lead to abrasion or even breakage of the ribs.

In all embodiments of the ribs, it is preferred that the possible contact area between the ribs and the medical device passed through the intravascular device be made as small as possible, so that, for example, in the case of semicircular ribs, a rounded side of the semicircle faces the medical device and not the flat base of the semicircle.

The inner liner has an inner liner inner diameter, for example in the range from 0.75 to 13 mm, and an inner liner outer diameter, for example in the range from 1 to 13.5 mm. Depending on the application, the inner liner can have different lengths. For example, the inner liner has a length in the range from 30 cm to 300 cm.

The inner liner inner diameter refers to the diameter of the inner liner without the ribs.

An outer jacket is coaxially arranged around the inner liner. The outer jacket is preferably the radially outer layer of the intravascular device, or at least of the tube-like body of the intravascular device, and serves in particular to mechanically protect the intravascular device. Preferably, the outer jacket has an outer diameter in a range from 200 μm to 5000 μm, more preferably from 300 μm to 3000 μm and most preferably from 500 μm to 1500 μm. In an exemplary embodiment, the outer diameter of the outer jacket is approximately 700 μm. It is understood that the outer diameter of the outer jacket can also correspond to the outer diameter of the intravascular device. The outer jacket preferably has a wall thickness in the range from 2 μm to 300 μm, particularly preferably from 5 μm to 150 μm and most preferably from 20 μm to 100 μm. According to a preferred embodiment of the present invention, the outer jacket has an outer diameter in a range from 300 μm to 3000 μm, most preferably from 500 μm to 1500 μm, and a wall thickness in a range from 5 μm to 150 μm, most preferably from 20 μm to 100 μm. In an exemplary embodiment, the wall thickness is approximately 60 μm. The outer jacket preferably consists of a thermoplastic polymer selected from the group of silicones, polyolefins (e.g. polyethylene), polyether block amide (e.g. PEBAX©), polyurethanes, polyimides, polyamides, polyaryletherketones (e.g. polyether ether ketone), fluorinated polymers (e.g. selected from the group of ethylene tetrafluoroethylene, polytetrafluoroethylene, perfluoroalkoxyalkanes, polyvinylidene fluorides, fluorinated ethylene propylene and mixtures thereof) and mixtures thereof. According to a preferred embodiment, the outer jacket comprises a polymer selected from the group consisting of silicones, polyolefins (e.g. polyethylene), polyurethanes, polyimides, polyamides, polyaryletherketone (e.g. polyetheretherketone), fluorinated polymers (e.g. selected from the group of ethylene tetrafluoroethylene, polytetrafluoroethylene, perfluoroalkoxyalkanes, polyvinylidene fluorides, fluorinated ethylene propylene and mixtures thereof) and mixtures thereof, and has an outer diameter in the range of 300 to 3000 μm.

In one embodiment, the inner liner and the outer jacket are joined to one another, at least in sections, in a materially cohesive manner, for example by melting together. Preferably the outer jacket comprises a polymer, preferably a thermoplastic polymer.

Since it is preferable to manufacture the intravascular device by the inner liner and the outer jacket, there should a material bond between these two layers. The materials of the inner liner and outer jacket should therefore be compatible wherever possible. If the materials are incompatible, a joining layer can be applied to the outer side of the inner liner and/or the inner side of the outer jacket, which enables a joining with a material bond of the two layers when heated. Preferably, such a joining layer is applied to the outer side of the inner liner. Such a joining layer is sometimes referred to as a “tie layer.” In addition to one or more joining layers, an outer surface of the corresponding layers can be treated to improve the joining between the inner liner and the outer jacket. For example, the outer surface of the inner liner can be etched or treated with plasma to increase its surface area.

The inner liner can be made of a variety of different materials, whereby the use of a flexible material or a flexible combination of materials is preferred. For example, the inner liner is made of a polymer selected from the group of silicones, polyolefins (e.g. polyethylene), polyether block amide (e.g. PEBAX©), polyurethanes, polyimides, polyamides, polyaryletherketones (e.g. polyether ether ketone), fluorinated polymers (e.g. selected from the group of ethylene tetrafluoroethylene, polytetrafluoroethylene, perfluoroalkoxyalkanes, polyvinylidene fluorides, fluorinated ethylene propylene and mixtures thereof) and mixtures thereof. According to a preferred embodiment, the inner liner comprises a polymer selected from the group consisting of silicones, polyolefins (e.g. polyethylene), polyether block amide (e.g. PEBAX©), polyurethanes, polyimides, polyamides, polyaryletherketone (e.g. polyether ether ketone), and mixtures thereof.

In a preferred embodiment of the intravascular device, the inner liner is made of a non-fluorinated thermoplastic polymer. Preferably, the inner liner comprises a polymer selected from the group consisting of silicones, polyolefins (e.g. polyethylene), polyether block amide (e.g. PEBAX©), polyurethanes, polyimides, polyamides, polyaryletherketone (e.g. polyether ether ketone), and mixtures thereof. More preferably, the inner liner consists of a polymer selected from the group consisting of silicones, polyolefins (e.g. polyethylene), polyether block amide (e.g. PEBAX©), polyurethanes, polyimides, polyamides, polyaryletherketone (e.g. polyether ether ketone), and mixtures thereof.

The number of ribs can be selected depending on the inner diameter of the inner liner respectively the diameter of the inner lumen as well as the height and width of the ribs and their spacing.

In a preferred embodiment of the intravascular device, the inner liner inner surface comprises 6 to 30, preferably 8 to 28, more preferably 10 to 26, ribs extending axially form the proximal end to the distal end of the inner liner.

The dimension, especially the height, of the ribs can be selected depending on the inner diameter of the inner liner respectively the diameter of the inner lumen, the number of ribs as well as the width of the ribs and their spacing.

In a preferred embodiment of the intravascular device, the ribs have a height that is between 0.012 mm and 0.08 mm. The height refers to the radial distance between the part of the ribs that extends furthest into the inner lumen and the base of the corresponding rib when viewed in cross-section. The base of the rib can be seen as an imaginary line between valleys adjacent to the respective ribs when viewed in cross-section.

A height in the range mentioned above preferably ensures that the medical device to be passed through the inner liner comes into contact only with the ribs and not with the valleys between the ribs, which could lead to tilting. Tilting could both make it more difficult to pass a medical device through the inner liner and increase the risk of abrasion or even breakage of the ribs.

In a preferred embodiment of the intravascular device, the ribs are evenly spaced around the circumference of the inner liner inner surface. Preferably, all ribs have the same dimensions, in particular the same shape und height. More preferably, all ribs are evenly spaced around the circumference of the inner liner inner surface and have the same dimensions, in particular the same shape und height. This could facilitate the manufacture of the intravascular device, in particular the manufacture of the inner lining, and minimize the risk of the medical devices becoming stuck inside the inner liner, in particular between adjacent ribs.

The spacing of the ribs can be selected depending on the inner diameter of the inner liner respectively the diameter of the inner lumen, the number of ribs as well as the dimensions, in particular the width and/or height, of the ribs.

In a preferred embodiment of the intravascular device, a maximum distance between two adjacent ribs is 0.5 mm, preferably 0.4 mm, more preferably 0.3 mm. The maximum distance is the distance between the points of the adjacent ribs in a plane perpendicular to the longitudinal axis of the inner liner that protrude the furthest into the inner lumen. In other words, this is the distance between the “tips” or “highest points” of the adjacent ribs when viewed in cross section. Such spacing can minimize the risk of a medical device getting stuck between adjacent ribs.

The ribs can be formed on the inner liner inner surface in different ways. For example, the ribs can be attached to the inner liner inner surface, for example by gluing.

In a preferred embodiment of the intravascular device, the ribs are integrally formed with the inner liner. This could facilitate the manufacture of the intravascular device, in particular the manufacture of the inner liner, and minimize the risk of the ribs accidentally detaching from the inner liner inner surface when using the intravascular device, which could result in a blockage of the inner lumen or even the introduction of at least parts of the ribs into the patient's blood vessels.

In a preferred embodiment of the intravascular device, the ribs are parallel to each other. In this embodiment, the ribs extend substantially parallel to one another or, to put it another way, they do not cross each other. This could minimize the risk of the medical device passing through the intravascular device getting stuck at the crossing of the ribs, which could lead to abrasion or even breakage of the ribs.

In a preferred embodiment of the intravascular device, the ribs extend in a straight or spiral pattern along the inner liner inner surface. In this embodiment, the ribs extend substantially parallel to one another in a straight manner, i.e. parallel to the longitudinal axis of the inner liner, or in a spiral manner, i.e. in helical turns around the inner circumference of the inner liner. However, in both embodiments, the ribs extend parallel to one another, which could minimize the risk of the medical device passing through the intravascular device getting stuck at the crossing of the ribs, which could lead to abrasion or even breakage of the ribs.

In a preferred embodiment of the intravascular device, the intravascular device comprises a reinforcement structure positioned between the inner liner and the outer jacket. A reinforcement structure is used in particular for the mechanical reinforcement of the intravascular device, and in particular also to be able to rotate it better around its longitudinal axis. The reinforcement structure thus transfers a torque or a rotation along the longitudinal axis of the intravascular device. For example the reinforcement structure is a coil or a series of coils, in particular a metal coil, a braid, in particular a metal braid, a wire construction, in particular a metal wire construction, a laser-cut hypotube, certain polymer layers, or any other known structure that can provide strength to allow for transmission of rotation or torque along the length of the intravascular device, in particular to the tube-like body of the intravascular device.

Preferably, the inner liner and the outer jacket are bonded to one another, for example they are fused or melted together, so that the reinforcement structure is fixed between the two layers. For this purpose, the reinforcement structure preferably has feedthroughs, for example in the form of windings in the case of reinforcement structures in the form of a coil or in the form of free spaces in the case of reinforcement structures in the form of a braid, through which the inner liner and the outer jacket are bonded to one another or are bonded in the course of the manufacturing process of the intravascular device.

The reinforcement structure can comprise different materials or consist of different materials. Preferably, the reinforcement structure comprises a polymer, a metal, or consists of a polymer or a metal. Examples of suitable polymers are polyether ether ketones (PEEK). Examples of suitable metals are titanium and alloys such as nickel-titanium alloys and stainless steel-in particular, 304V or 316I stainless steel.

A further subject matter of the invention relates to a method of manufacturing an intravascular device with ribs, in particular an intravascular device according to the forementioned embodiments of the invention, comprising the method steps:
(a) extruding a hollow cylindrical inner liner having a proximal end, a distal end, and an inner lumen extending axially through the inner liner between the proximal end and the distal end, wherein the inner lumen is surrounded by an inner liner inner surface;
(b) simultaneously forming a plurality of ribs on the inner liner inner surface during the extrusion process, the ribs extending axially from the proximal end to the distal end of the inner liner; and
(c) arranging an outer jacket coaxially around the inner liner.

In method steps (a) and (b), which occur simultaneously, the inner liner and the ribs on the inner liner inner surface are produced though an extrusion method. Extrusion is a simple and cost-effective process and provides a simple and cost-effective way of forming the ribs in various orientations, dimensions and shapes. In this method, the ribs become an integral part of the inner liner. Preferably, the ribs are made of the same material as the inner liner, ideally the same thermoplastic polymer or polymer blend.

In method step (c) the outer jacket is coaxially arranged around the inner liner. This can be achieved in various ways. For instance, the outer jacket can be formed by sleeving a hollow cylindrical heat shrink tube onto the inner liner, whereby the heat shrink tube, after being sleeved onto the inner liner, contracts radially when heated and forms the outer jacket. This embodiment contains a reflow process that shrinks the heat shrink tube.

Preferably, the outer jacket is also applied using an extrusion process. The extrusion process of the outer jacket contains a reflow process for at least soften or even melt the material of the outer jacket. The extrusion of the outer jacket in process step (c) can take place simultaneously with process steps (a) and (b).

To place a reinforcement structure between the inner liner and the outer jacket, an appropriate structure can be added to the inner liner in an intermediate step between method steps (b) and (c). This reinforcement structure is then preferably encased along with the inner liner by the outer jacket in process step (c). For instance, in this intermediate step, a coil or braid can be arranged around the inner liner.

Depending on the type, in particular the dimension and shape of the ribs, as well as the material of the ribs and/or the entire inner liner, it may be preferable to support the ribs formed in method step (b) during subsequent steps to, at least largely retain, their shape and dimension. This is particularly advantageous if the ribs or the entire inner liner are made of a material, such as a thermoplastic polymer, with a melting temperature below the temperature that might be applied in subsequent steps, such as in method step (c).

In a preferred embodiment of the method for manufacturing an intravascular device, wherein arranging the outer jacket in method step (c) comprises a reflow process, a structured mandrel is placed in the inner lumen, the structured mandrel having a pattern corresponding to the ribs of the inner liner, thereby preserving the ribs on the inner liner inner surface during the reflow process. The structured mandrel thus has indentations that correspond to the shape and dimensions of the ribs of the inner liner, so that the ribs are inserted into the indentations of the mandrel before the reflow process takes place. This way, the ribs remain at least largely in their original form if the reflow process is carried out at temperatures that would soften or even melt the material of the ribs and/or the inner liner. Without the structured mandrel, softening or melting of the ribs could lead to deformation or even the disappearance of the ribs. The structured mandrel can be removed from the inner lumen after the reflow process.

A further subject matter of the invention relates to a method of manufacturing an intravascular device with ribs, in particular an intravascular device according to the forementioned embodiments of the invention, comprising the method steps:
(a) providing a hollow cylindrical inner liner having a proximal end, a distal end, and an inner lumen extending axially through the inner liner between the proximal end and the distal end, wherein the inner lumen is surrounded by an inner liner inner surface;
(b) placing a structured mandrel in the inner lumen, the structured mandrel having a pattern corresponding to the desired ribs;
(c) heating the inner liner to a temperature sufficient to cause the inner surface to melt, or at least to soften, and conform the pattern of the structured mandrel, thereby forming a plurality of ribs on the inner liner inner surface, the ribs extending axially form the proximal end to the distal end of the inner liner; and
(d) arranging an outer jacket coaxially around the inner liner.

The inner liner can be provided in various ways in method step (a). For example, the inner liner can be provided by means of an extrusion process.

The structured mandrel provided in method step (b) has indentations that correspond to the shape and dimensions of the intended ribs of the inner liner.

In method step (c), the inner liner is heated so that at least its inner surface softens enough to allow it to be placed into the indentations of the structured mandrel. This can be done, for example, by capillary action or gravity alone. Preferably, the insertion of the inner liner into the indentations of the structured mandrel is assisted by a force acting radially inwards, so the inner liner inner surface is preferably pressed into the pattern of the structured mandrel. After removal of the structured mandrel, preferably after cooling of the inner liner, the inner liner is provided with the desired ribs.

In method step (d) the outer jacket is coaxially arranged around the inner liner. This can be achieved in various ways. For instance, the outer jacket can be formed by sleeving a hollow cylindrical heat shrink tube onto the inner liner, whereby the heat shrink tube, after being sleeved onto the inner liner, contracts radially when heated and forms the outer jacket. This embodiment contains a reflow process that shrinks the heat shrink tube.

Preferably, the outer jacket is applied using an extrusion process. The extrusion process of the outer jacket contains a reflow process for at least soften or even melt the material of the outer jacket.

To place a reinforcement structure between the inner liner and the outer jacket, an appropriate structure can be added to the inner liner in an intermediate step between method steps (a) and (d). This structure is then preferably encased along with the inner liner by the outer jacket in process step (d). For instance, in this intermediate step, a coil or braid can be arranged around the inner liner.

The features disclosed in the claims, the specification, and the drawings maybe essential for different embodiments of the claimed invention, both separately and in any combination with each other.

### FIGURES

The invention is further illustrated by way of example below by means of figures. The invention is not limited to the figures.

The following are shown

FIG. 1 a schematic cross-section through an exemplary intravascular device comprising an inner liner with ribs,

FIG. 2 a schematic cross-section of another exemplary intravascular device comprising an inner liner with ribs,

FIG. 3 a schematic cross-section of another exemplary intravascular device comprising an inner liner with ribs,

FIG. 4 a flowchart of an exemplary method for manufacturing an intravascular device comprising an inner liner with ribs, and

FIG. 5 a flowchart of a further exemplary method for manufacturing an intravascular device comprising an inner liner with ribs.

### DESCRIPTION OF THE FIGURES

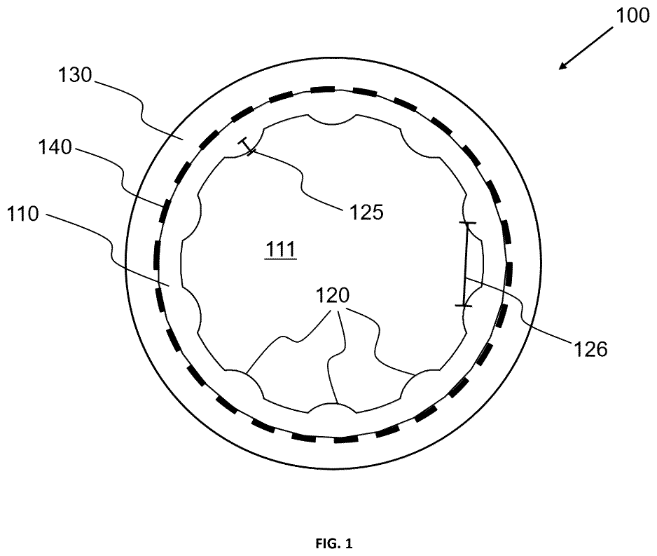

FIG. 1 shows a cross-section of an exemplary intravascular device 100, in particular a cross-section through a tube-like body of an exemplary intravascular device 100, for introducing a medical device into the vascular system of a patient. The intravascular device 100 comprises a hollow cylindrical inner liner 110, through which an inner lumen 111 extends axially from a proximal end to a distal end of the inner liner 110. The inner lumen 111 serves to insert an medical device into the patient when the intravascular device 100 is in use. In order to effectively reduce a contact surface between the inner liner 110 and the medical device to be passed through the inner lumen 111, an inner liner inner surface comprises a plurality, in the shown embodiment ten, of axially extending parallel ribs 120 (only exemplarily provided with reference signs). The ribs 120 are an integral part of the inner liner 110 and have a rounded cross-section to minimize the contact between the inner liner 110 and the medical device to be passed through the inner lumen 111. The ribs 120 have a height 125 of 0.01 mm and a distance 126 of 0.4 mm between two adjacent ribs 120. A reinforcement structure 140 is arranged and fixed between the inner liner 110 and the outer jacket 130. In the embodiment shown, the reinforcement structure 140 is a metallic braid. The inner liner 110 and the outer jacket 130 are bonded to each other in areas without the reinforcement structure 140, so that the reinforcement structure 140 is fixed between the two layers 110, 130.

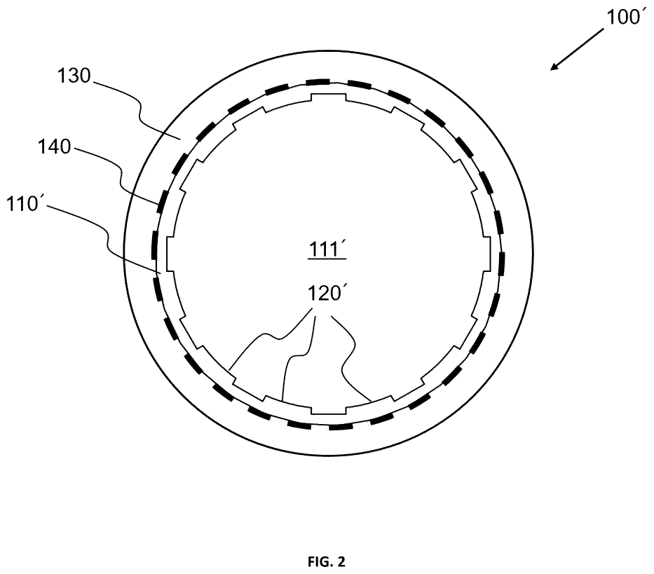

FIG. 2 shows a cross-section of another exemplary intravascular device 100′. The embodiment according to FIG. 2 is largely consistent with the embodiment described above and shown in FIG. 1, so that, to avoid repetition, reference is made to the above description. A structure that is repeated from the description of FIG. 1 has the same reference sign. Modifications to the structure shown in FIG. 1 have the same reference sign with an additional apostrophe.

The inner liner 110′ of the intravascular device 100′ comprises 12 ribs, which, in contrast to the embodiment shown in FIG. 1, are angular in shape.

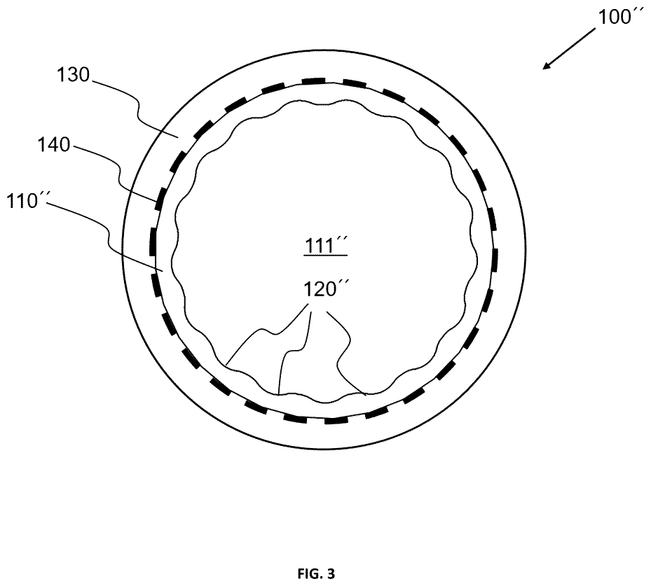

FIG. 3 shows a cross-section of another exemplary intravascular device 100″. The embodiment according to FIG. 3 is largely consistent with the embodiment described above and shown in FIG. 1, so that, to avoid repetition, reference is made to the above description. A structure that is repeated from the description of FIG. 1 has the same reference sign. Modifications to the structure shown in FIG. 1 have the same reference sign with two additional apostrophes.

The inner liner 110″ of the intravascular device 100″ has 17 rounded ribs, which, in contrast to the embodiment in FIG. 1, are closer together, so that the ribs 120″ occupy substantially the entire circumference of the inner liner 110″.

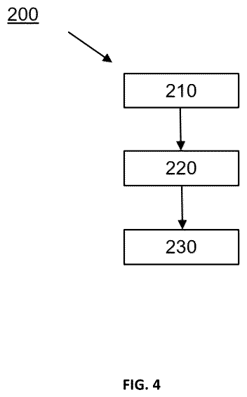

FIG. 4 shows a flowchart of an exemplary method 200 for manufacturing an intravascular device 100, 100′, 100″, comprising the method steps 210 to 230.

In method step 210, the hollow cylindrical inner liner 110, 110′, 111″ is extruded, with the inner lumen 111, 111′, 111″ extending axially through it.

Simultaneously with method step 210, method step 220 takes place, in which the plurality of ribs 120, 120′, 120″ are formed on the inner liner inner surface. The ribs 120, 120′, 120″ are thus also produced by extrusion in this embodiment of the method 200 and thus form an integral part of the inner liner 110, 110′, 111″. Preferably, the ribs 120, 120′, 120″ are made of the same material, preferably of the same thermoplastic polymer, as the rest of the inner liner 110, 110′, 111″. Preferably, this material is a non-fluorinated polymer.

In method step 230, the outer jacket 130 is coaxially arranged around the inner liner 110, 110′, 110″. This method step 230 may comprise a reflow process so that the inner liner 110, 110′, 110″ and the outer jacket 130 form a material bond and are thus firmly connected to one another. Furthermore, a reinforcement structure 140 may be arranged around the inner liner 110, 110′, 110″ before arranging the outer jacket 130.

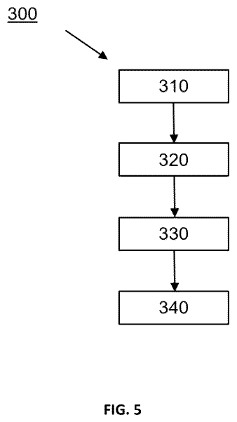

FIG. 5 shows a flowchart of another exemplary method 300 for manufacturing an intravascular device 100, 100′, 100″, comprising the method steps 310 to 340.

In method step 310, the hollow cylindrical inner liner 110, 110′, 110″ is provided, preferably by an extrusion process.

In method step 320, a patterned mandrel is placed in the inner lumen 111, 111′, 111″ of the inner liner 110, 110′, 110″ provided in method step 310, the patterned mandrel having a pattern that corresponds to the desired ribs 120, 120′, 120″. The pattern of the patterned mandrel is a negative of the ribs 120, 120′, 120″.

In method step 330, the inner liner 110, 110′, 110″ is heated to a temperature to cause the inner surface of the inner liner 110, 110′, 110″ to melt, or at least to soften, and conform the pattern the pattern of the structured mandrel, thereby forming the plurality of ribs 120, 120′, 120″.

In method step 340, the outer jacket 130 is coaxially arranged around the inner liner 110, 110′, 110″. This method step 340 may comprise a reflow process so that the inner liner 110, 110′, 110″ and the outer jacket 130 form a material bond and are thus firmly connected to one another. Furthermore, a reinforcement structure 140 may be arranged around the inner liner 110, 110′, 110″ before arranging the outer jacket 130.

### REFERENCE SIGNS

100 intravascular device
110 inner liner
111 inner lumen
120 rib
125 height
126 distance
130 outer jacket
140 reinforcement structure
200 method
210 extruding
220 forming
230 arranging
300 method
310 providing
320 placing
330 heating
340 arranging

1. An intravascular device for introducing medical devices into vascular systems, comprising:
a hollow cylindrical inner liner comprising a proximal end, a distal end, and an inner lumen extending axially through the inner liner between the proximal end and the distal end,
wherein the inner lumen is surrounded by an inner liner inner surface of the inner liner, an outer jacket arranged coaxially around the inner liner, and
wherein the inner liner inner surface comprising a plurality of ribs extending axially from the proximal end to the distal end of the inner liner.

2. The intravascular device according to claim 1, wherein the inner liner comprises a non-fluorinated thermoplastic polymer.

3. The intravascular device according to claim 1, wherein the inner liner inner surface comprises 6 to 30 ribs extending axially from the proximal end to the distal end of the inner liner.

4. The intravascular device according to claim 1, wherein the ribs have a height that is between 0.012 mm and 0.08 mm.

5. The intravascular device according to claim 1, wherein the ribs are evenly spaced around the circumference of the inner liner inner surface.

6. The intravascular device according to claim 1, wherein a maximum distance between two adjacent ribs is 0.5 mm.

7. The intravascular device according to claim 1, wherein the ribs are integrally formed with the inner liner.

8. The intravascular device according to claim 1, wherein the of ribs are parallel to each other.

9. The intravascular device according to claim 1, wherein the ribs extend in a straight or spiral pattern along the inner liner inner surface.

10. The intravascular device according to claim 1, further comprising a reinforcement structure positioned between the inner liner and the outer jacket.

11. The intravascular device according to claim 1, wherein the inner liner is made of a polymer selected from the group consisting of polyether block amide copolymers, polyamides, polyethylenes and polyimides.

12. A method of manufacturing an intravascular device with ribs, comprising:
(a) extruding a hollow cylindrical inner liner having a proximal end, a distal end, and an inner lumen extending axially through the inner liner between the proximal end and the distal end, wherein the inner lumen is surrounded by an inner liner inner surface;
(b) simultaneously forming a plurality of ribs on the inner liner inner surface during the extrusion process, the ribs extending axially from the proximal end to the distal end of the inner liner; and
(c) arranging an outer jacket coaxially around the inner liner.

13. The method according to claims 12, wherein arranging the outer jacket comprises a reflow process using a structured mandrel placed in the inner lumen, the structured mandrel having a pattern corresponding to the ribs on the inner liner, thereby preserving the ribs on the inner liner inner surface during the reflow process.

14. A method of manufacturing a intravascular device with ribs, comprising:
(a) providing a hollow cylindrical inner liner having a proximal end, a distal end, and an inner lumen extending axially through the inner liner between the proximal end and the distal end, wherein the inner lumen is surrounded by an inner liner inner surface;
(b) placing a structured mandrel in the inner lumen, the structured mandrel having a pattern corresponding to the desired ribs;
(c) heating the inner liner to a temperature sufficient to cause the inner surface to melt and conform the pattern of the structured mandrel, thereby forming a plurality of ribs on the inner liner inner surface, the ribs extending axially form the proximal end to the distal end of the inner liner; and
(d) arranging an outer jacket coaxially around the inner liner.

15. The method according to claims 12, wherein arranging the outer jacket comprises a reflow process using the structured mandrel placed in the inner lumen, thereby preserving the ribs on the inner liner inner surface.
